(19) United States
(12) Patent Application Publication
(10) Pub. No.: US 2008/0051821 A1
(43) Pub. Date: Feb. 28, 2008

(54) TISSUE DILATION TOOL AND METHOD OF DILATING TISSUE

(76) Inventor: Matthew P. Gephart, Marquette, MI (US)

Correspondence Address: FITCH EVEN TABIN AND FLANNERY, 120 SOUTH LA SALLE STREET, SUITE 1600, CHICAGO, IL, 60603-3406, US

(21) Appl. No.: 11/843,611

(22) Filed: Aug. 22, 2007

Related U.S. Application Data
(60) Provisional application No. 60/813,628, filed on Aug. 22, 2006.

(51) Int. Cl. A61M 29/00

(52) U.S. Cl. 606/191

(57) ABSTRACT

Tissue dilation tools and methods of use are disclosed that provide gradual and controlled stretching of the tissue surrounding a surgical incision. The tools utilize a plurality of operably connected, coaxial sleeves or members of different sizes to gradually enlarge the incision.

### FIELD

The invention relates generally to a tissue dilation tool and method of use, and more particularly to a multi-stage tissue dilation tool and method of use.

### BACKGROUND

While an incision into a patient's skin is often required during surgical procedures to gain access to a surgical site, such incisions can cause damage, injury, and trauma to the patient's body. Increased trauma to the body can undesirably lead to prolonged recovery time, infections and longer hospital stays following surgery. To avoid causing unnecessary trauma, it is preferable to make the incisions as small as possible, while also providing sufficient access to the surgical site. One way to accomplish these dual objectives is to make a smaller incision and then stretch the tissue surrounding the incision to form an enlarged opening providing sufficient access to the surgical site.

For example, to repair damage to a patient's spine, a surgeon may want to implant a spinal fixation device. To attach the implant to the patient's spine the surgeon will need to have access to the spine. While a surgeon could make one large incision to accommodate the spinal fixation procedure, it can be preferable for the surgeon to make multiple smaller incisions at the points of interest. The multiple smaller incisions can then be stretched to provide access to the surgical site by the tools and components necessary to implant the spinal fixation device. If multiple smaller incisions are made and then stretched, the patient's tissue can be exposed to less trauma.

There are a number of tools that are used to stretch the surrounding tissue around an incision, including series dilators and retractors. Series dilators, which are often used to stretch tissue surrounding an incision, are typically comprised of a number of separate tubes of different diameters each having a tapered end. The separate tubes of increasing diameter can be slid down one another with the tapered ends being inserted into the surgical incision to gradually stretch the tissue surrounding an incision. If the tubes are not used in sequentially increasing diameters, the tissue may be subject to tearing or blunt force trauma when the tubes are pushed into the incision because the surrounding tissue does not have the benefit of the gradually increasing diameter to gradually stretch the tissue. Standard dilation systems typically use a number of tapered tubes of increasing diameter. Thus, the series dilation procedure can require that the surgeon distinguish between the numerous independent tubes of different diameters to ensure incremental dilation of the tissue.

Accordingly, there remains a need for a tool that incrementally stretches, in a controlled manner, tissue surrounding an incision into an enlarged opening.

### SUMMARY

A tissue dilation tool and method of use are disclosed that provide gradual and controlled stretching of the tissue surrounding a surgical incision. The tool utilizes a plurality of operably connected, coaxial sleeves or members of different sizes to gradually enlarge the incision.

In a preferred form, each of the sleeves or members has a tapered end that is inserted into the incision and is used to gradually stretch the tissue surrounding the incision. The sleeves are in a telescoping arrangement and are operable to sequentially stretch the tissue surrounding the incision by having the tapered ends of sequentially larger diameters sleeves inserted into the incision to stretch the tissue surrounding the incision by an amount greater than that possible with the previous sleeve of the tool. To assist in the sequential insertion of the tapered ends of the sleeves into the incision, one or more of the sleeves can be selectively restricted from movement relative to other of the sleeves.

Before the tissue dilation tool is used, an incision is made into the patient's body where surgical access is desired. The incision is typically a small stab wound. Depending on surgeon preference, a guide wire may be placed into the incision before use of the tissue dilation tool. After making the incision, the tapered portion of the smallest diameter sleeve is first inserted into the incision. By having a relatively small diameter of the end of the taper enter the incision first, followed by the increasing diameter of the tapered portion, the skin around the incision is gradually stretched using each sleeve to create an enlarged opening that is sufficient to accommodate the next-larger diameter sleeve. Following use of the last of the sleeves, the incision is preferably enlarged to the desired size, such as to accommodate surgical instruments and components required to perform the remainder of the surgical procedure.

In order to facilitate sequential insertion of the sleeves or members into the incision, each of the members or sleeves can be locked relative to the other sleeves or members. Once a smaller diameter sleeve has been used to stretch the incision, it can be released from the other sleeves to permit the smaller diameter sleeve to slide within the next larger diameter sleeve. This will permit the tapered end of the next larger diameter sleeve to be inserted into the opening and used to further enlarge the opening. After the smallest diameter sleeve has been sufficiently advanced into the surgical site, the lock is released or disengaged such that the next largest diameter sleeve can be advanced down the smaller diameter sleeve and into the incision. Thus, the next largest diameter sleeve can enter the incision and further stretch the tissue by forcing the tissue to accommodate the increasing diameter of the next sleeve. This process continues until the largest sleeve has been advanced into the incision and/or the incision has been sufficiently dilated.

In a preferred form, a single manually-operable lock can be used to control collapsing of the sleeves. The single manually-operable lock can function with an automatic lock that engages when adjacent sleeves are in particular relative arrangements.

The tissue dilation tool may include an inner member, an intermediate sleeve and an outer sleeve, each of increasing diameter and having tapered ends. The tissue dilation tool is locked in a fully extended position when the tapered end of the inner member is inserted into the incision. By having the inner member locked in the extended position, the force from the tissue surrounding the incision does not cause the collapse of the tissue dilation tool at the skin's surface. Instead, the surgeon is able to push the tissue dilation tool into the incision and thereby incrementally stretch the tissue. Next, the inner member can be unlocked to permit the intermediate sleeve to slide over the inner member and into the enlarged incision. Finally, the intermediate sleeve can be unlocked to permit the outer sleeve to slide over both the intermediate sleeve and inner member and into the further enlarged incision.

The tissue dilation tool can preferably be disassembled such that the dilation tool can easily be cleaned after use. The largest diameter sleeve may have an attachment on the end opposite the tapered portion. The attachment may function to permit disassembly of the tissue dilation tool when removed. When present, the attachment may function to limit the sliding movement of the smaller sleeves to prevent them from separating from the larger sleeve.

### BRIEF DESCRIPTION OF THE DRAWINGS

FIG. 1 is a front elevation view of a tissue dilation tool showing the inner member and intermediate sleeve in their fully extended positions relative to the outer sleeve;

FIG. 2 is a cross-sectional view of the tissue dilation tool of FIG. 1;

FIG. 3 is an enlarged view of the tissue dilation tool of FIG. 2 showing a locking mechanism between the inner member and the outer sleeve in a locked position;

FIG. 4 is an enlarged view of a portion of the tissue dilation tool of FIG. 2 showing a locking mechanism between the intermediate sleeve and the outer sleeve in a locked position;

FIG. 5 is a front elevation view of the tissue dilation tool of FIG. 1 showing the intermediate sleeve in its fully extended position and the inner member in a partially retracted position relative to the outer sleeve;

FIG. 6 is a cross-sectional view of the tissue dilation tool of FIG. 5;

FIG. 7 is an enlarged view of the tissue dilation tool of FIG. 6 showing the locking mechanism between the inner member and the outer sleeve in an unlocked position;

FIG. 8 is an enlarged view of the tissue dilation tool of FIG. 6 showing the locking mechanism between the intermediate sleeve and the outer sleeve in the locked position;

FIG. 9 is a front elevation view of the tissue dilation tool of FIG. 1 showing the intermediate sleeve in its fully extended position and the inner member in its fully retracted position relative to the outer sleeve;

FIG. 10 is a cross-sectional view of the tissue dilation tool of FIG. 9;

FIG. 11 is an enlarged view of the tissue dilation tool of FIG. 10 showing the locking mechanism between the inner member and the outer sleeve in the locked position;

FIG. 12 is an enlarged view of the tissue dilation tool of FIG. 10 showing the locking mechanism between the intermediate sleeve and the outer sleeve in an unlocked position;

FIG. 13 is a front elevation view of the tissue dilation tool of FIG. 1 showing the intermediate sleeve and the inner member in their fully retracted positions relative to the outer sleeve;

FIG. 14 is a cross-sectional view of the tissue dilation tool of FIG. 13 showing the locking mechanism between the intermediate sleeve and the outer sleeve in the locked position;

FIG. 15 is an enlarged view of the tissue dilation tool of FIG. 14 showing the locking mechanism between the inner member and the outer sleeve in the locked position;

FIG. 16 is an enlarged view of the tissue dilation tool of FIG. 14 showing the locking mechanism between the intermediate sleeve and the outer sleeve in the unlocked position;

FIG. 17 is an exploded sectional view of the tissue dilation tool of FIG. 1;

FIG. 18 is a perspective view of an alternative tissue dilation tool in an extended position;

FIG. 19 is a perspective view of the tissue dilation tool of FIG. 18 in a collapsed position;

FIG. 20 is a cross-sectional view of the tissue dilation tool of FIG. 18 in the collapsed position;

FIG. 21 is a cross-sectional view of an alternative tissue dilation tool showing the inner member and intermediate sleeve in their fully extended positions relative to the outer sleeve;

FIG. 22 is an enlarged view of a portion of the tissue dilation tool of FIG. 21 showing a locking mechanism between the inner member and the outer sleeve in a locked position;

FIG. 23 is an enlarged view of a portion of the tissue dilation tool of FIG. 21 showing a locking mechanism between the intermediate sleeve and the outer sleeve in a locked position;

FIG. 24 is a cross-sectional view of the tissue dilation tool of FIG. 21 showing the intermediate sleeve in its fully extended position and the inner member in a partially retracted position relative to the outer sleeve;

FIG. 25 is an enlarged view of a portion of the tissue dilation tool of FIG. 24 showing the locking mechanism between the inner member and the outer sleeve in an unlocked position;

FIG. 26 is an enlarged view of a portion of the tissue dilation tool of FIG. 24 showing the locking mechanism between the intermediate sleeve and the outer sleeve in the locked position;

FIG. 27 is a cross sectional view of the tissue dilation tool of FIG. 21 showing the intermediate sleeve in its fully extended position and the inner member in its fully retracted position relative to the outer sleeve;

FIG. 28 is an enlarged view of a portion of the tissue dilation tool of FIG. 27 showing the locking mechanism between the inner member and the outer sleeve in the locked position;

FIG. 29 is an enlarged view of a portion of the tissue dilation tool of FIG. 27 showing the locking mechanism between the intermediate sleeve and the outer sleeve in the unlocked position;

FIG. 30 is a cross sectional view of the tissue dilation tool of FIG. 21 showing the intermediate sleeve and the inner member in their fully retracted positions relative to the outer sleeve;

FIG. 31 is an enlarged view of a portion of the tissue dilation tool of FIG. 30 showing the locking mechanism between the inner member and the outer sleeve in the locked position;

FIG. 32 is an enlarged portion of the tissue dilation tool of FIG. 30 showing the locking mechanism between the intermediate sleeve and the outer sleeve in the unlocked position;

FIG. 33 is an exploded view of the tissue dilation tool of FIG. 21;

FIG. 34 is a cross sectional view of another alternative tissue dilation tool showing the inner member and intermediate sleeve in their fully extended positions relative to the outer sleeve;

FIG. 35 is an exploded view of the tissue dilation tool of FIG. 34;

FIG. 36 is a cross sectional view of another alternative tissue dilation tool showing the inner member and intermediate sleeve in their fully extended positions relative to the outer sleeve; and

FIG. 37 is an exploded view of the tissue dilation tool of FIG. 36.

### DETAILED DESCRIPTION

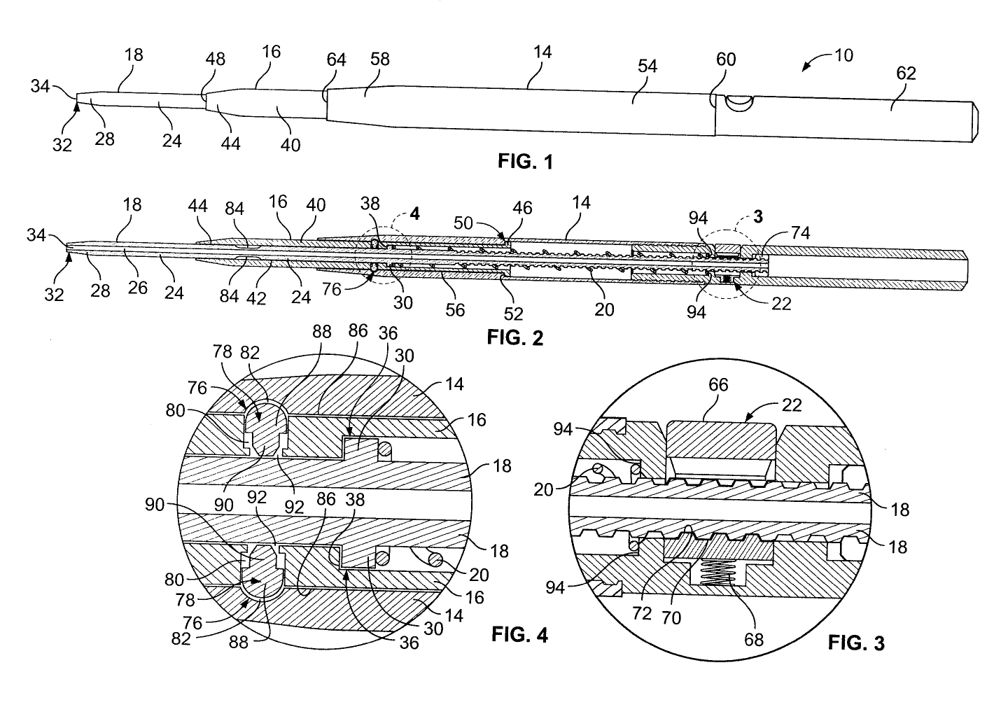

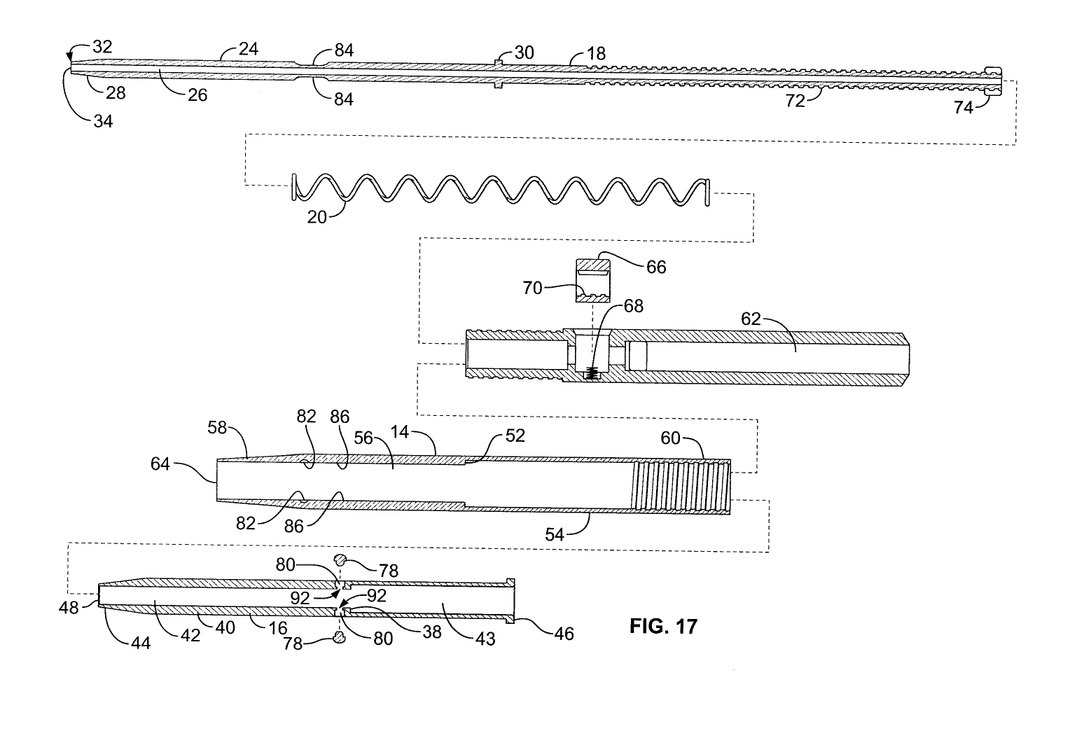

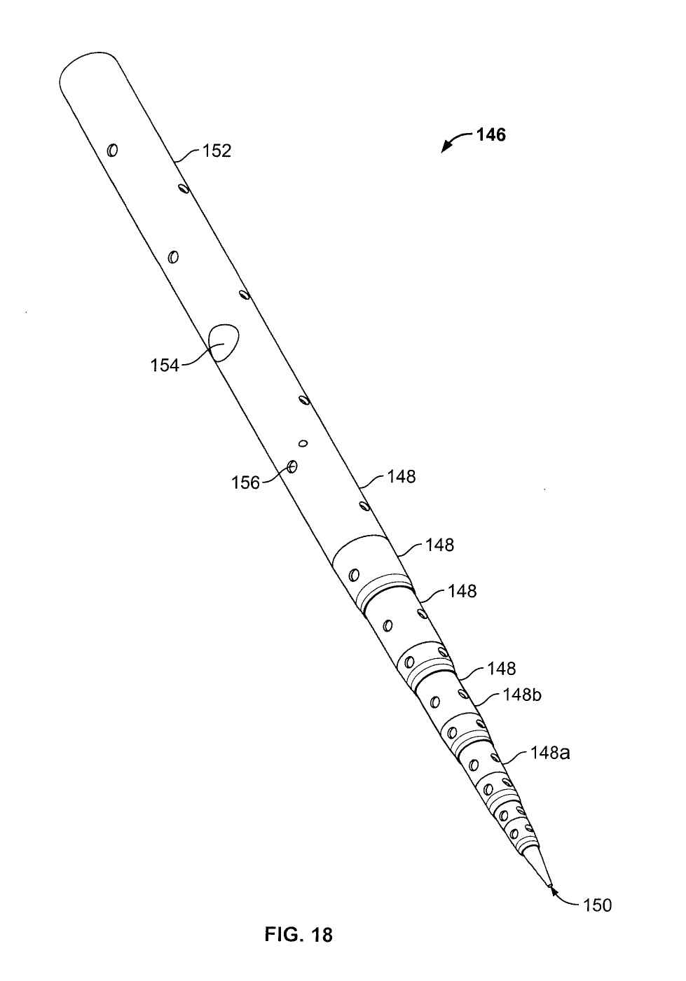

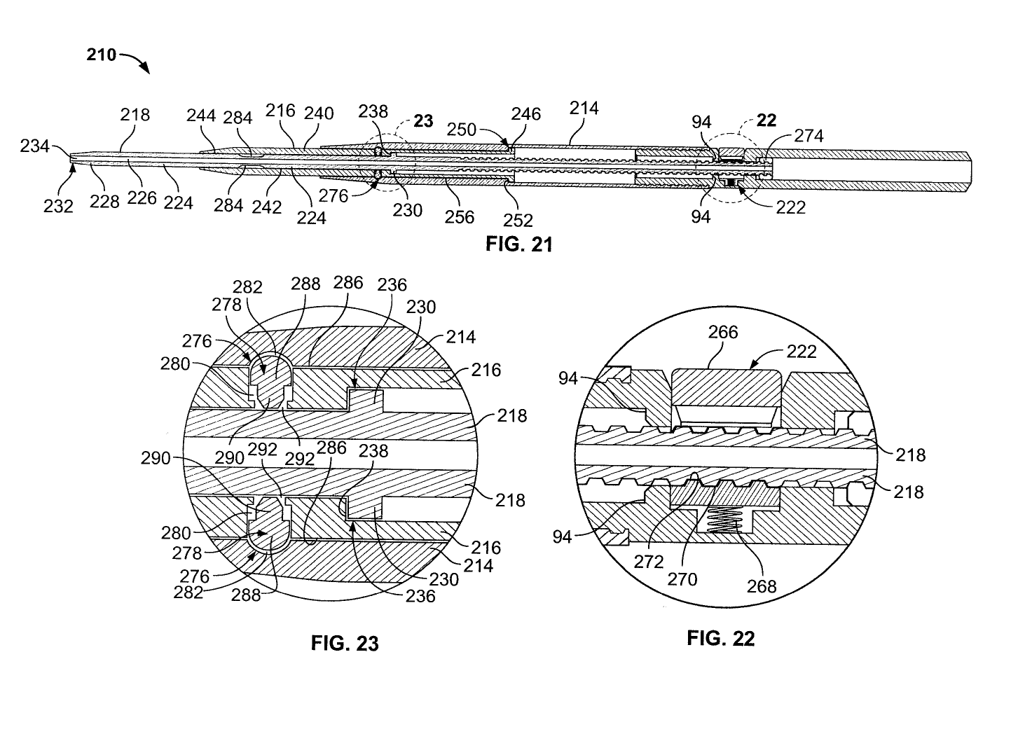

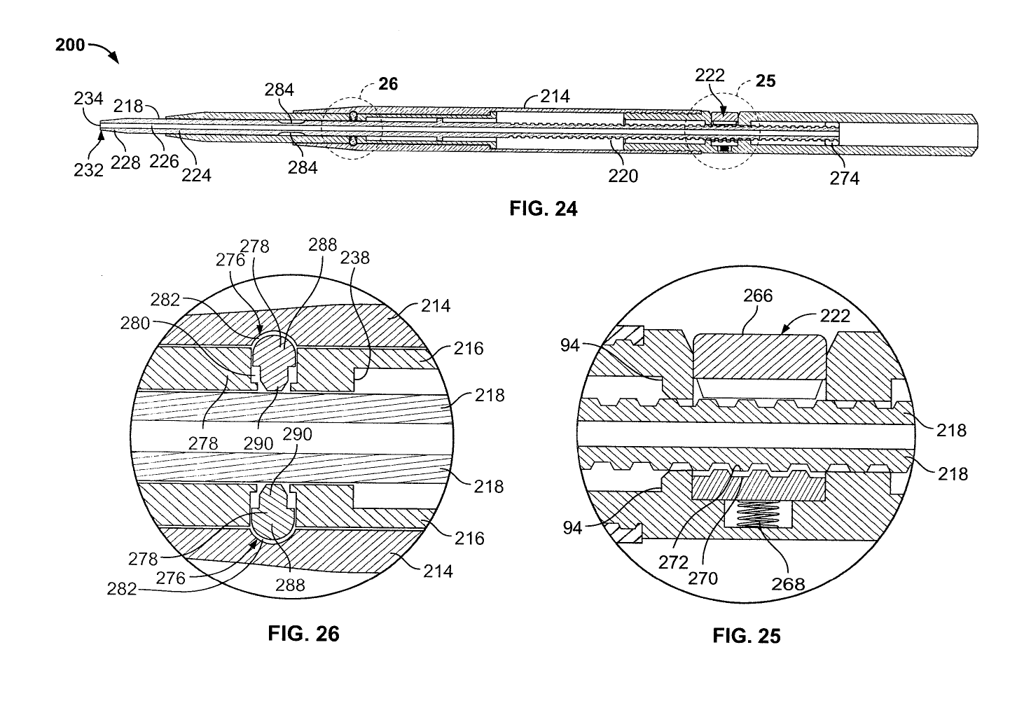

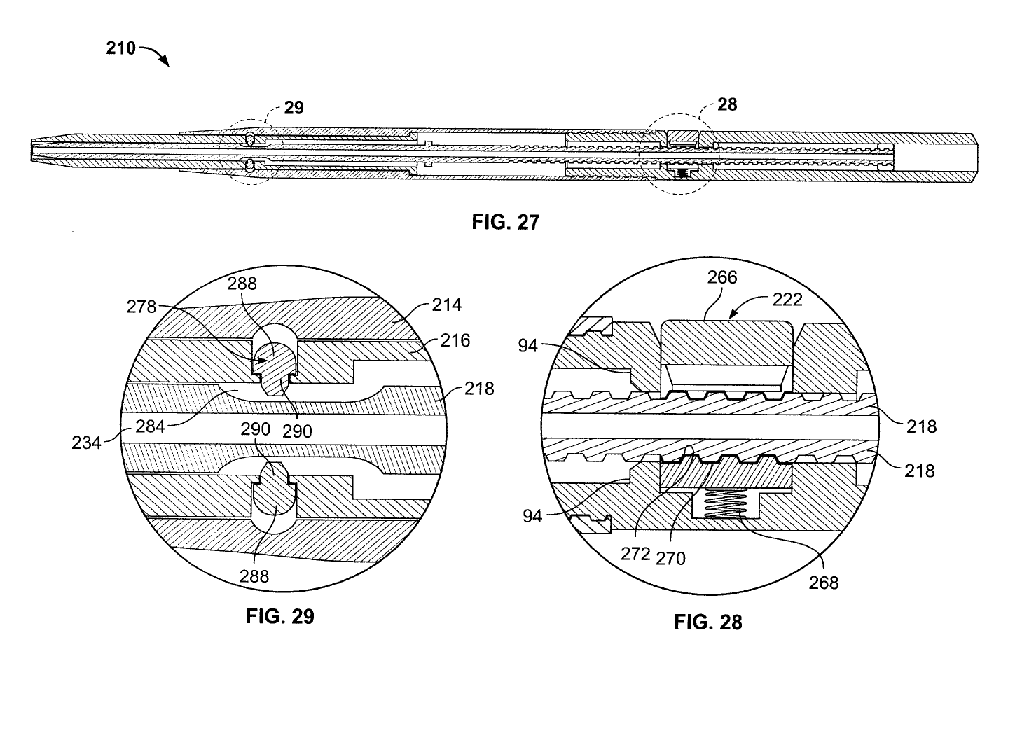

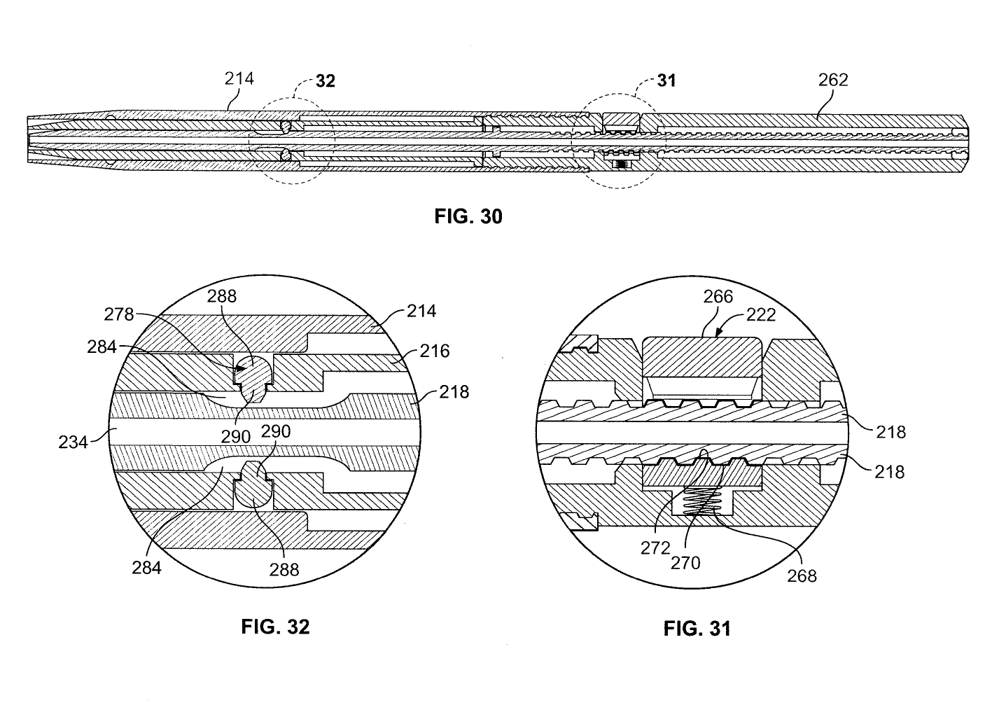

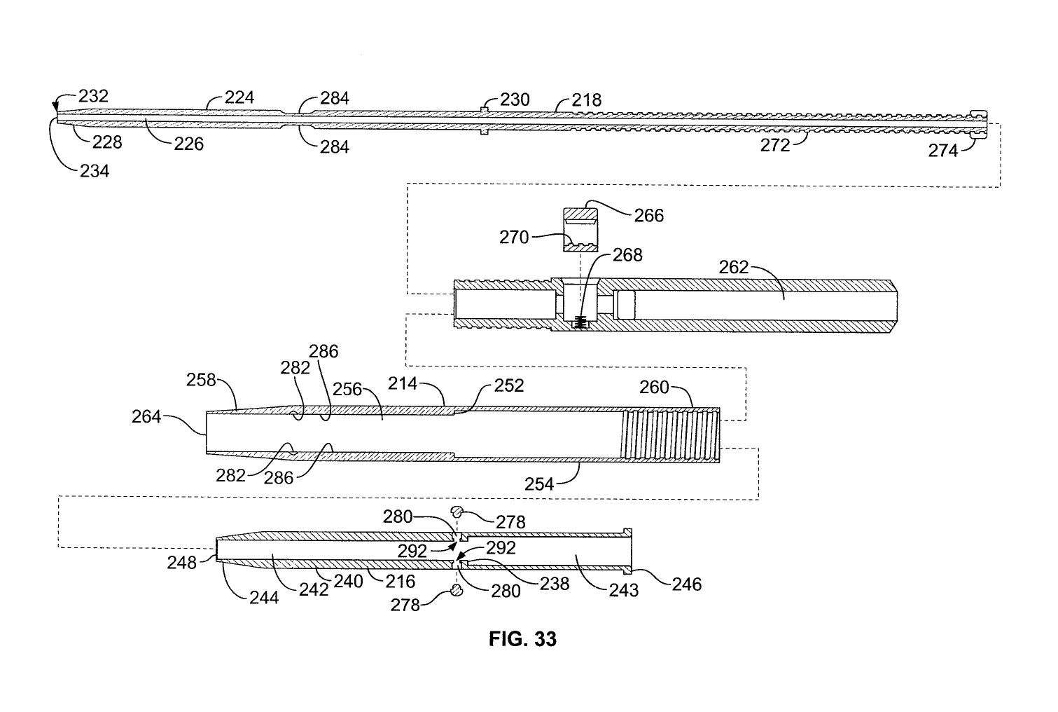

Tissue dilation tools for use in sequentially enlarging a surgical incision are disclosed herein and illustrated in FIGS. 1-33. The tissue dilation tools utilize multiple stages and a plurality of telescoping tissue engaging members in order to incrementally stretch the tissue surrounding an incision. The telescoping tissue dilation tools generally include a number of sleeves or members having tapered end portions facing in the same direction. The sleeves of the tissue dilation tools can be independently moved between an extended position and a retracted position relative to other sleeves to permit the sleeves to be used sequentially in order of increasing diameter. In one embodiment, illustrated in FIGS. 1-17, the sleeves are biased toward and can be locked in their extended positions relative to an outer sleeve to prevent inadvertent collapsing of the tissue dilation tool. In an alternative embodiment, illustrated in FIGS. 18-20, a plurality of the sleeves are independently biased to extended positions using associated springs. In yet another alternative embodiment, illustrated in FIGS. 21-33, the sleeves can be locked in their extended positions relative to an outer sleeve to prevent inadvertent collapse of the tissue dilation tool, but are not biased toward the extend position.

In the illustrated embodiment of FIGS. 1-17, an inner member 18, an intermediate sleeve 16 and an outer sleeve 14 are telescopingly arranged. The inner member 18 is slidable within the intermediate sleeve 16 and the intermediate sleeve 16 is slidable within the outer sleeve 14. The inner member 18 can be locked relative to the outer sleeve 14 and the intermediate sleeve 16 can be locked relative to the outer sleeve 14. The use of multiple locks and multiple sleeves or members facilitates the sequential use of increasing diameter sleeves to dilate an incision in a multi-stage dilation process.

When the tissue dilation tool 10 is advanced into the incision, the dilation tool 10 remains in the completely extended position, with each of the inner member 18, intermediate sleeve 16 and outer sleeve 14 secured relative to one another in a first stage. After the smallest diameter inner member 18 has sufficiently advanced into the incision, the two larger diameter sleeves 16 and 14 are advanced down around the inner member 18. As the intermediate sleeve 16 is advanced down the inner member 18, the intermediate sleeve 16 enters the incision in a second stage, followed by the largest diameter outer sleeve 14 in a third stage. Each of the inner member 18, intermediate sleeve 16 and outer sleeve 14 are pushed into the incision against the force of the tissue so that the tissue around the incision stretches and creates a larger working channel without requiring a larger incision into the patient.

In a first stage of the dilation process, shown in FIGS. 1-4, the inner member 18, intermediate sleeve 16 and outer sleeve 14 are secured relative to one another in their fully extended positions. The tapered portion 28 of the inner member 18 of the fully extended dilation tool 10 can be advanced into the incision during the first stage to incrementally stretch the tissue surrounding the incision to accommodate an increasing diameter of the tapered portion 28 of the inner member 14. In the first stage, locking mechanisms 22 and 76 serve to retain the inner member 18 and the intermediate sleeve 16 in their extended positions despite the force of the tissue against the inner member 18. More specifically, the inner member 18 is locked relative to the outer sleeve 14 using an inner member locking mechanism 22 and the intermediate sleeve 16 is locked relative to the outer sleeve 14 using an intermediate sleeve locking mechanism 76.

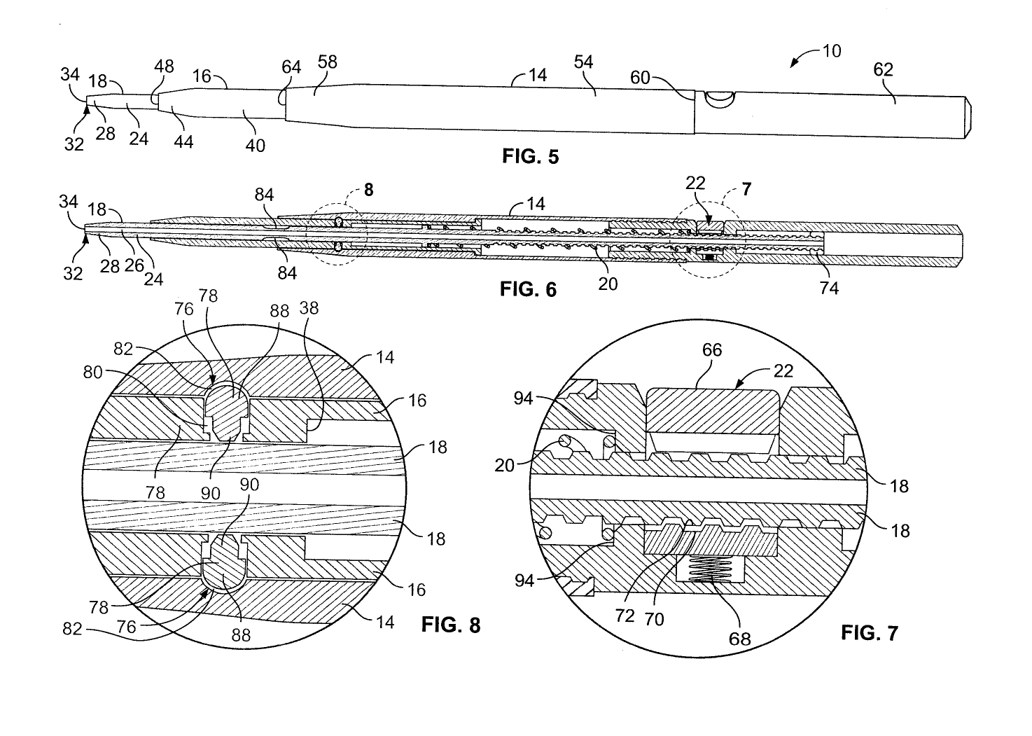

After the tapered end 28 of the inner member 18 has been sufficiently advanced into the incision, the inner member locking mechanism 22 can be released, as illustrated in FIGS. 5-7, to allow the inner member 18 to move to its retracted position within the intermediate sleeve 16 for use in a second stage of the tissue dilation process. In the second stage of tissue dilation, the intermediate sleeve 16 remains fixed relative to the outer sleeve 14 using the intermediate sleeve locking mechanism 76, as illustrated in FIG. 8. During the second stage, a tapered end 44 of the intermediate sleeve 16 is advanced down the inner member 18 and into the incision to incrementally stretch to accommodate the increasing diameter of the tapered end 44 of the intermediate sleeve 16. To encourage a controlled collapse of only the inner member 18, the intermediate sleeve locking mechanism 76 limits the movement of the intermediate sleeve 16 relative to the outer sleeve 14 during the second stage.

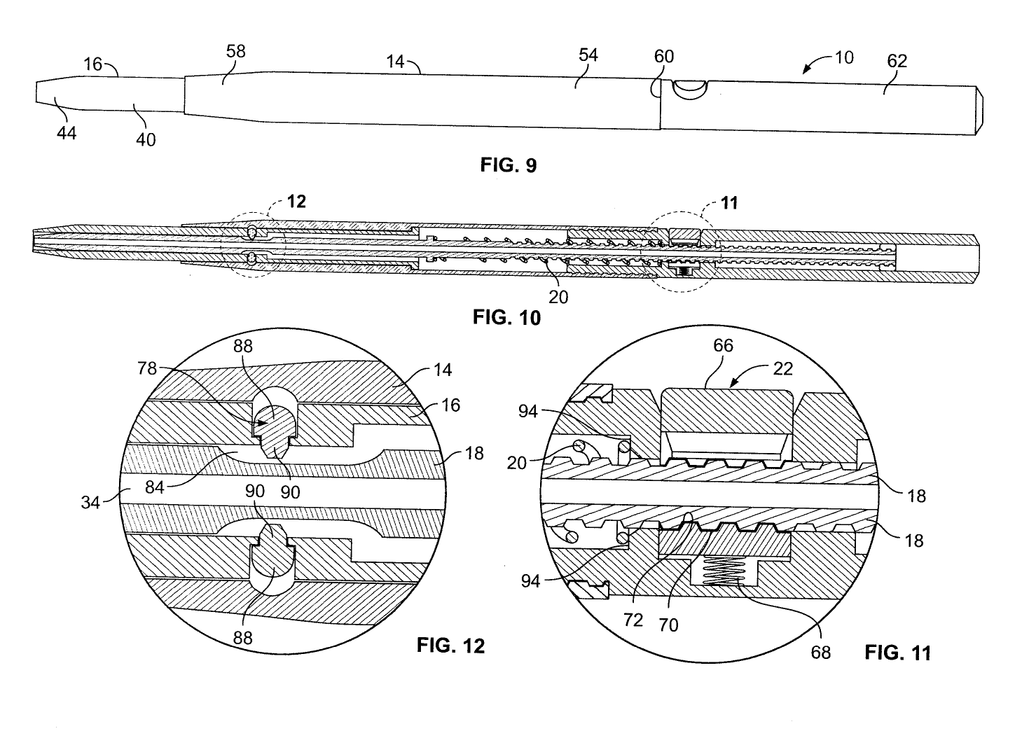

In a third stage of the process of tissue dilation using the tissue dilation tool 10, the outer sleeve 14 is advanced down the intermediate sleeve 16 and into the incision, thereby continuing to stretch the tissue surrounding the original incision and dilate the surgical opening. Similar to the inner member 18 and intermediate sleeve 16, the largest diameter outer sleeve 14 includes a tapered portion 58 that can incrementally stretch the tissue. As illustrated in FIGS. 9-12, the inner member locking member 22 is engaged to limit movement of the inner member 18 relative to the outer sleeve 14, which in turn limits movement of the intermediate sleeve 16 relative to the inner member 18 via the locking mechanism 76, as will be described in greater detail below. Thus, the inner member 18 and the intermediate sleeve 16 travel may travel together into the outer sleeve 14 as the outer sleeve 14 is advanced down the intermediate sleeve 16.

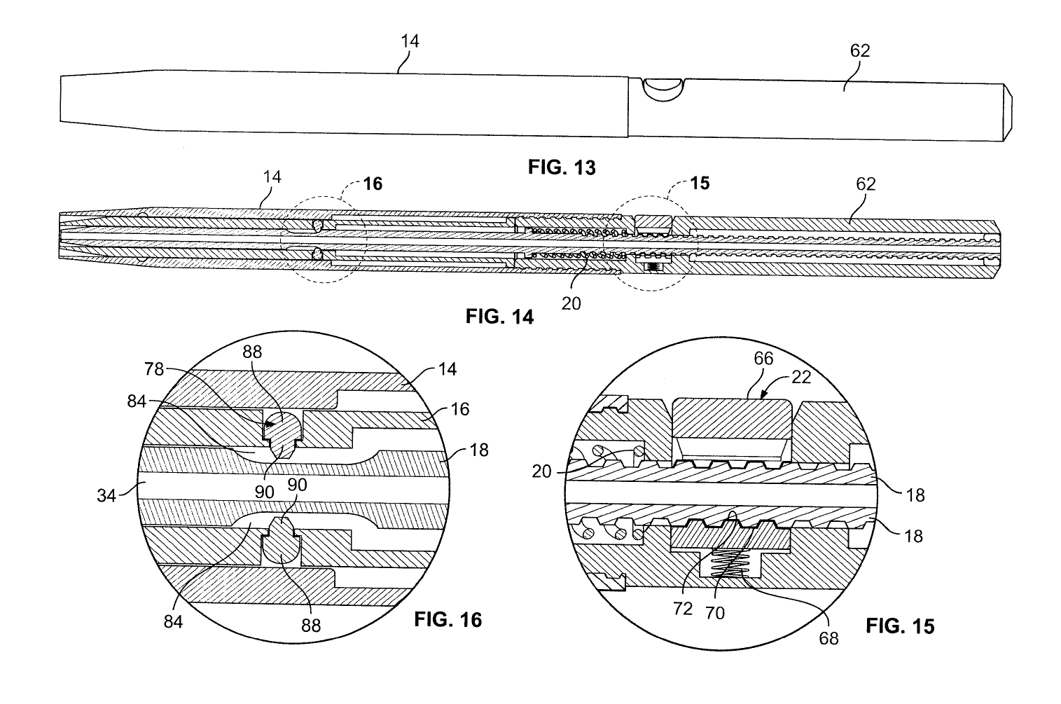

When the inner member 18 and the intermediate sleeve 16 are in a fully retracted position, illustrated in FIGS. 13-16, the inner member locking mechanism 22 and intermediate sleeve locking mechanism 76 are engaged to restrict movement of the inner member 18 and intermediate sleeve 16 relative to the outer sleeve 14.

The tissue dilation tool 10 is placed inside the previously established surgical incision and may be placed over a guide wire if the surgeon so chooses. To accommodate the guide wire, the sleeves may be cannulated thereby allowing the tissue dilation tool 10 to be used with a guide wire. If a guide wire is not employed, the inner member 18 may be a solid cylindrical body instead of a cannulated sleeve. The sleeves are telescoped together, the inner member 18 having the smallest diameter, the intermediate sleeve 16 having the next smallest diameter, and the outer sleeve 14 having the largest diameter of the three sleeves.

The telescoped sleeves are biased toward an extended position by a spring 20 and secured into position relative to one another by the inner member locking mechanism 22. In the fully extended position, at least the tapered portion 28 of the inner member 18 is fully projected from the tapered portion 44 of the intermediate sleeve 16. Similarly, at least the tapered portion 44 of the intermediate sleeve 16 is fully projected from the tapered portion 58 of the outer sleeve 14. If the lock 22 has been released and the biasing force from spring 20 is overcome, the telescoping tissue dilation tool 10 begins to collapse to a retracted position. The collapse begins with the inner member 18 being advanced into the tapered portion of the intermediate sleeve 16. The tissue dilation tool 10 has a number of mechanisms that create a controlled collapse, such that the outer sleeve 14, intermediate sleeve 16, and inner member 18 move relative to one another in a sequential manner.

Turning now to FIGS. 1 and 2, the inner member 18 includes a general cylindrical portion 24 with an inner bore 26 for receiving a guide wire, the tapered portion 28, and a flanged portion 30. The tapered portion 28 converges to a nose 32 having an opening 34 that provides access to the inner bore 26. However, as mentioned above, the inner member 18 may also be a solid cylinder with a tapered end similar to an obturator. As the tapered portion 28 is advanced into the incision the skin surrounding the incision stretches to accommodate the increasing diameter of the tapered portion 28. The tapered portion 28 illustrated is conical with a gradual slope, thereby incrementally stretching the tissue as the diameter of the tapered portion 28 increases, however, other slopes are possible and can be selected to either stretch the tissue more abruptly or more gradually.

During the second stage, the inner member 18 is movable within the intermediate sleeve 16. A stop mechanism 36 is provided between the inner member 18 and the intermediate sleeve 16 to limit the sliding movement of the inner member 18 outwardly from the intermediate sleeve 16. In the illustrated embodiment, the stop mechanism 36 includes the radially-extending flange 30 located on the inner member 18 and a shelf 38 or radial should located on the intermediate sleeve 16. The flange 30 engages the shelf 38 when the inner member 18 is in the fully extended position. Conversely, the flange 30 can engage the housing 62 to function as a stop limiting retraction of the inner member 18 relative to the outer sleeve 14, which is attached to the housing 62.

The intermediate sleeve 16 has a general cylindrical portion 40 with an inner bore 42 such that the intermediate sleeve 16 can receive the inner member 18 and guide wire, if present. The intermediate sleeve 16 also has a tapered portion 44 at one end, and a radially extending flange 46 at the other end. The smallest diameter of the tapered portion 44 and the inner diameter of the intermediate sleeve 16 are both slightly larger than the largest outside diameter of the inner member 18. This provides for clearance such that the inner member 18 can slide within the intermediate sleeve 16. If sufficient tolerance is not provided, the sleeves may translate the force being exerted thereupon, such that the inner member 18 causes damage to the bone. Further, if the guide wire is present it may be pushed into or through the bone causing injury. By having the smallest outer diameter of the tapered portion 44 slightly larger than the largest outer diameter of the inner member 18, the tissue is gradually stretched as the next sleeve enters the incision. The gradual increase in the external diameter of the sleeves allows the surgeon to gradually stretch the soft tissue. The tapered portion 44 has an opening 48 that provides access to the inner bore 42. While a conical tapered portion with a gradual slope is illustrated, the tapered portion 44 could also have a differently shaped.

The intermediate sleeve 16 is located partially within the outer sleeve 14 in the extended position and has its outward movement limited by a stop mechanism 50 between the outer sleeve 14 and the intermediate sleeve 16. In the illustrated embodiment, the stop mechanism 50 includes the flange 46 that abuts a shelf or radial shoulder 52 located inside the inner bore 42 of the outer sleeve 14 when the intermediate sleeve 16 is biased outwardly from the outer sleeve 14. The shelf 52 is located at the point where the inner bore 42 expands to a larger diameter bore 40.

As can be seen in FIG. 2, the outer sleeve 14 is a hollow tube with a generally cylindrical portion 54 having an inner bore 56 for receiving the intermediate sleeve 16, the inner member 18, and the guide wire, if present. The outer sleeve 14 also includes a tapered portion 58 and an attachment end 60 that fastens a handle housing 62 to the outer sleeve 14. The smallest diameter of the tapered portion 58 and the inner diameter of the outer sleeve 14 are both slightly larger than the largest outside diameter of the intermediate sleeve 16. The clearance provided by such dimensions allows the sleeves 14 and 16 to easily slide relative to one another. Further, by having the smallest diameter of the tapered portion 58 only slightly larger than the largest diameter of the intermediate sleeve 16, the tissue is gradually stretched as the next sleeve enters the incision. The tapered portion 58 has an opening 64 that extends to the inner bore 56. While a conical tapered portion with a gradual slope is illustrated, the tapered portion 58 could also have a different shape.

The outer sleeve 14 is disposed around the intermediate sleeve 16 and inner member 18. The housing 62 is located on the end of the outer sleeve 14 opposite the tapered portion 58 and limits movement of the intermediate sleeve 16 and inner member 18, as discussed above. When removed from the outer sleeve 14, the housing 62 allows the intermediate sleeve 16 and inner member 18 to be released from the outer sleeve 14. Thus, to disassemble the tissue dilation tool 10 for cleaning, the housing 62 is removed from the outer sleeve 14. The housing 62 can be attached to the outer sleeve 14 by a threaded connection, press fit, or other suitable connection.

During use, the tapered portion 28 of the inner member 18 first enters the surgical incision. The tapered portion 28 is first advanced into the patient's body. The surgeon overcomes the resistance of the tissue by pushing and/or rotating the tissue dilation tool 10 depending on the surgeon's preferred practice. The inner member 18 remains locked or fixed in the extended position by the lock 22. After the inner member 18 has sufficiently entered the surgical incision, the surgeon can manually release or disengage the lock 22 thereby allowing the outer sleeve 14 and intermediate sleeve 16 to move with respect to the inner member 18. Then, while lock 22 is kept in the disengaged position and while applying an axial longitudinal force toward the nose 32 of the tapered portion 28, the outer sleeve 14 and intermediate sleeve 16 advance together down the inner member 18.

In one illustrated embodiment, the lock 22 is located in the housing and includes a button 66 and spring 68. The button 66 has threads 70 that engage threads 72 located on the inner member 18. The threads 70 and 72 are preferably Acme threads, for example 12-24 threads UNC 2A Acme threads. The Acme threads typically have a roughly 29 degree pitch with a flat apex and valley, which can be stronger than the typical V-profile 60 degree thread. When the button 66 is pushed, the threads 72 on the inner member 18 disengage the button threads 70 thereby allowing the inner member 18 to move relative to the outer sleeve 14. The spring 68 pushes the button 66 radially outward such that the button threads 70 remain engaged with the threads 72 of the inner member 18. When the button 66 is depressed, the threads 70, 72 disengage and when the button 66 is released, the threads 70, 72 reengage and stop the movement between the inner member 18 and the outer sleeve 14. An enlarged nut 74 located on the end of the inner member 18 opposite the tapered portion 28 can limit the movement of the inner member 18 outwardly relative to the outer sleeve 14 upon abutment which a shelf or ledge in a bore of the housing 62. Depending upon where the nut 74 is positioned on the threads 72 of the inner member 18, as well as the relative positions of the inner member 18, outer sleeve 14 and intermediate sleeve 16, either engagement of the nut 74 with the shelf of the housing 62 or the stop 30 can limit outward extension of the inner member 18.

To assure sequential collapsing of the sleeves, the tissue dilation tool 10 is equipped with mechanisms to stop the sliding movement of the sleeves with the mechanisms including a selectively engagable lock 76. The selectively engagable lock 76 has a primary position, shown in FIG. 4, in which the sliding movement of the outer sleeve 14 is stopped with respect to the intermediate sleeve 16 so that sleeve 14, 16 can only slidingly move together with respect to the inner member 18 and an unlocked position, shown in FIG. 12, in which the outer sleeve 14 can slidingly move relative to the intermediate sleeve 16. In addition, the selectively engagable lock 76 has a secondary locked position, shown in FIG. 14, where the plunger 78 is used to limit relative movement between the inner member 18 and intermediate sleeve 16.

In one embodiment, the selectively engagable lock 76 includes at least one plunger or locking member 78 and preferably four plungers or locking members 78 incorporated into the intermediate sleeve 16. The plunger 78 resides in a stepped aperture 80 located in the intermediate sleeve 16. The plunger 78 can protrude into a recess 82 located in the outer sleeve 14 or can protrude into a recess 84 located in the inner member 18. When the plunger is protruding into the recess 82 of the outer sleeve 14, the locking mechanism 76 is typically in the primary position whereby sliding movement of the outer sleeve 14 is stopped with respect to the intermediate sleeve 16. When the plunger is protruding into the recess 84 of the inner member, the locking mechanism is typically in the secondary position whereby sliding movement is limited between the inner member 18 and intermediate sleeve 16.

When the inner member 18 and the intermediate sleeve 16 are moved to their retracted positions relative to the outer sleeve 14, the plunger 78 is initially held in the recess 82 of the outer member 14 by the generally cylindrical portion 24 of the inner member 18. Once the inner member 18 has been advanced sufficiently into the intermediate sleeve 16 to bring the recess 84 into alignment with the plunger 78, further movement of the outer sleeve 14 relative to the intermediate sleeve 16 pushes the plunger 78 into the recess 84 in the inner member 18 by interaction between camming surfaces of the recess 82 of the outer member and camming surfaces of the plunger 78. When the outer sleeve 14 is continued to be advanced down the intermediate sleeve 16, a flat longitudinally extending inner wall 86 of the outer sleeve 14 keeps the narrow portion 90 of the plunger 78 protruding into the recess 84 of the inner member 18 as shown in FIG. 16. Further advancement of the inner member 18 relative to the intermediate sleeve 16 can cause the plunger 78 to ride to an end of the recess 84, as illustrated in FIG. 14, whereby engagement between the plunger 78 and the end of the recess 84 limits further movement in that direction and the locking mechanism is in the secondary lock position.

When the inner member 18 and the intermediate sleeve 16 are moved to their extended positions relative to the outer sleeve 14, such as when the locking mechanism 22 is disengaged to permit the spring 20 to bias the inner member 18 outwardly, the plunger 78 is initially held in the recess 84 of the inner member 18 by the inner wall 86 of the outer sleeve 14. Biasing of the inner member 18 outwardly also causes biasing of the intermediate sleeve 16 outwardly due to engagement between the plunger 78, held in the intermediate sleeve 16, and an end of the recess 84 of the inner member. Once the inner member 18 and intermediate sleeve have together been advanced so that the plunger 78 is aligned with the recess 82 of the outer sleeve 14, further advancement of the inner member 18 due to the biasing of the spring 20 causes camming surfaces of the end of the recess 84 to engage with camming surfaces of the plunger 78 to urge the plunger 78 radially outwardly from the inner member 18 and into the recess 82 of the outer sleeve 14, where it is held in the primary lock position once the plunger 78 is abutted by the generally cylindrical portion 24 of the inner member, thereby limiting movement of the outer sleeve 14 relative to the intermediate sleeve 16. The inner member 18 can then be biased outwardly relative to both the intermediate sleeve 16 and the outer sleeve 14 by the spring 20.

In one embodiment, the plunger 78 includes a dome-shaped portion 88 and a post 90. The plunger 78, located in the stepped aperture 80 has the dome-shaped portion 88 oriented to face the outer sleeve 14 and the post 90 oriented to face toward the inner member 18. The post 90 is located within a through-hole 92 of the stepped aperture 80. When the inner wall 86 maintains the plunger 78 radially inward, the post 90 is positioned within the inner member recess 84. When the plunger 78 is not within the inner member recess 84 but rather the cylindrical portion 24, the dome-shaped portion 88 extends into the outer sleeve recess 82. In the fully extended position, the dome shaped portion 88 remains in the outer sleeve recess 82, however, when the outer sleeve 14 begins advancing down the intermediate sleeve 16 the longitudinally extending flat inner wall 86 pushes the plungers to the secondary position.

Both recesses 82 and 84 located on the outer member 14 and inner member 18, respectively, have portions that cammingly engage the plungers 84, as discussed generally above. The camming portion of the recess 82 is arcuate and can cam against the outward camming surface of the dome-shaped portion 88, thereby pushing the post portion 90 into recess 82 when urged thereagainst. The camming portions of recess 84 may also function as stops, as shown in FIG. 14 and discussed above, to limit the movement of the intermediate sleeve 16 and inner member 18 relative one another when the locking mechanism 76 is in the secondary lock position.

The spring 20 biases the inner member 18 and intermediate sleeve 16 to the fully extended position. In a preferred embodiment, the spring 20 is located in the inner bore 56 of the outer sleeve 14 and the spring 20 extends around the inner member 18. One end of the spring 20 engages the flanged portion 30 of the intermediate sleeve 16 and the other end engages a stop 94 in housing 62. In this manner the spring 20 pushes the intermediate sleeve 16 to the fully extended position. When the housing 62 is removed from the outer sleeve 14, the spring can be removed from the outer sleeve 14 and cleaned along with the intermediate sleeve 16 and inner member 18.

In the illustrated embodiment, the sleeves are each cannulated for using the tissue dilation tool 10 with a guide wire. The guide wire is preferably 14 inches long and has a diameter of about 0.065 inches depending on the size of the inner bore. If a guide wire is employed, and the surgeon has established the initial stab wound, the surgeon inserts the guide wire into the incision and advances the guide wire to the position of interest, possible at a boney location where an implant is to be anchored. Some surgeons utilize imaging equipment to assist them with correct placement of the guide wire into the patient. Use of imaging equipment, such as fluoroscopic tools, helps prevent the surgeon from misplacing the guide wire or pushing the guide wire through or even beyond the walls of the bone. The preferred guide wire is self-cutting with a self-tapping thread, although this too may change with surgeon preference. Typical guide wires are constructed of biocompatible metals or alloys such as nitinol, stainless steel, or titanium.

While the tissue dilation tool 10 is suited for a number of dimensions, it is contemplated that in one configuration the largest diameter of the outer sleeve 14 will be around 0.633 inches. The outer sleeve 14 tapers to a smaller diameter of preferably 0.453 inches. It is preferable to have a shelf area at the tapered end 58 of the outer sleeve 14 that is inserted into the patient so that the sleeve 14 does not have a sharp point. The inner diameter of the outer sleeve 14 is about 0.433 inches. The inner diameter of the outer sleeve 14 is sized to easily slide over the largest diameter, approximately 0.428 inches, of the intermediate sleeve 16. The smallest diameter of the intermediate sleeve 16 is about 0.230 inches. The inner diameter of the intermediate sleeve 16 must be larger than the 0.215 inches, which is the largest diameter of the inner member 18. The smallest diameter of the inner member 18 is 0.071 inches.

An alternative dilation tool is disclosed in U.S. Provisional Patent Appl. Nos. 60/722,604, filed on Sep. 29, 2005, and 60/655,983, filed on Feb. 23, 2005, and International Appl. No. PCT/US06/06684, filed on Feb. 23, 2006, the disclosures of which are hereby incorporated by reference in their entireties. The alternative dilation tool 146, illustrated in FIGS. 18-20, includes multiple sleeves each independently biased into an extended position by an associated spring. The springs have sequentially increasing spring forces, which together permit the sequential retraction of the sleeves or members in order from smallest diameter to largest diameter.

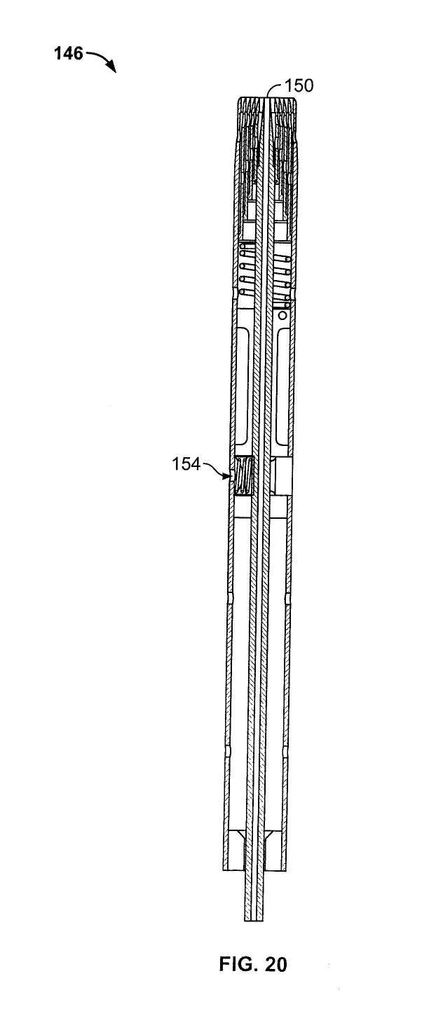

More specifically, the harpoon dilator 146 comprises a series of progressively larger spring-loaded, interdependent sleeves or cylinder portions 148. Each interdependent cylinder 148 is preferably biased away from the next cylinder by a series of springs into an extended position, an example of which is illustrated in FIG. 18. Although only one spring is shown for purposes of clarity in FIG. 20, it will be understood that a separate spring can be associated with each cylinder to either bias the cylinder from the adjacent inner cylinder or from a common spring stop. Such springs have a progressively higher spring constant as the diameter of the independent cylinder portions 148 increases. The dilator 146 may further include a reduced diameter nose 150 that is cannulated to allow for passage of a guidewire.

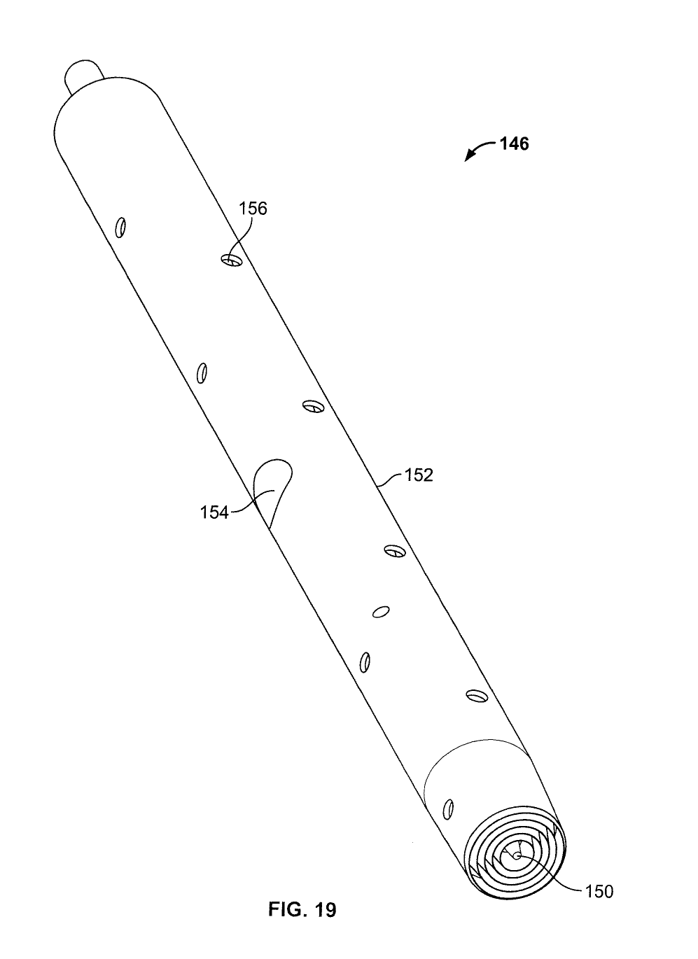

The harpoon dilator 146 can be placed over the guidewire 132 by placing the end of the guidewire into the cannulated nose 150. The dilator 146 may then be slid down the guidewire until the nose 150 contacts the bone surface. The surgeon then drives the extended dilator toward the bone, progressively dilating the soft tissue by pushing a smaller diameter cylinder 148a into an adjacent, larger diameter cylinder 148b. When the final cylinder 152, which is also the largest, has been pushed into contact with the surrounding soft tissue and contacts or closely approximates the bone, the surrounding tissue has been stretched to the diameter of the largest cylinder 152. When the harpoon dilator has been fully collapsed it automatically locks into the configuration, as illustrated in FIGS. 19 and 20, using a spring-biased locking mechanism. To extend the harpoon dilator to its original fully extended configuration, a release button of the locking mechanism 154 may be depressed.

The harpoon dilator 146 can be removed from the system either in the collapsed or extended position by sliding it back off the guidewire. The harpoon dilator 146 could be configured to stretch the tissue without the assistance of the guidewire, and may be include cleansing holes 156 for facilitating instrument sanitation.

The sleeves are illustrated having a circular cross-section, but other cross-sections can be used, such as ovoid or hexagonal. While the tissue dilation tool depicted in the drawings has three sleeves, tissue dilation tools having more than three sleeves can be used. In addition, the tissue dilation tools can have different numbers of sleeves, cylinders or members, and the use of the terms outer, intermediate and inner is relative to those three elements. For example, an exterior sleeve may surround the outer sleeve, and an interior member may be disposed within the inner member.

In another embodiment, a tissue dilation tool 210 includes members or sleeves that are operably connected by their telescoping configuration and various structures such as locking mechanism and stops but without the assistance of a spring to bias the sleeves to the extended position, like spring 20 of tissue dilation tool 10. The alternative embodiment is illustrated in various stages of operation in FIGS. 21-33. In these figures, parts which are similar to those previously discussed in FIGS. 1-17 are similarly numbered with the exception of having a prefix “2”. The tool 210 includes an inner member 218, an intermediate sleeve 216, and an outer sleeve 214 arranged and configured such that the relative movement between them occurs in a controlled sequential manner.

As shown in FIGS. 21-33, the tissue dilation tool 210 lacks the spring 20 shown in tool 10. Removing the spring 20 removes a component from such tool 210 such that cleaning and assembly may be made easier and quicker. Without spring 20, the collapse and relative movement of the inner member 218, the intermediate sleeve 216, and the outer sleeve 214 are controlled by a locking mechanism 222 and a selectively engageable lock 276. More specifically, the locking mechanism 222 is used to secure or fix the inner member 218 relative to the outer sleeve 214 and the locking mechanism 276 is used to secure or fix the intermediate sleeve 216 relative to the outer sleeve 214. In addition, enlarged stop or nut 274 stops the sliding movement of the inner member 218 at its fully extended position protruding out from the end of the intermediate sleeve 216 such that it is not fully discharged from intermediate sleeve 216 and outer sleeve 214.

The inner member 218, the intermediate sleeve 216, and the outer sleeve 214, similar to previous embodiments, include tapered ends 228, 244, 258 respectively. The tapered ends 228, 244, 258 are the portions of the tool that are inserted into the surgical site. During the dilation procedure, the tapered end of the smallest diameter tube (inner member 218) is inserted and advanced into the surgical site, followed by the next smallest diameter tube (intermediate sleeve 216). After the inner member 218 has been sufficiently advanced into the wound, the locking mechanism 222 between the inner member 218 and the outer sleeve 214 is released or disengaged as by depressing button 266 against its spring bias such that the intermediate sleeve 216 along with the outer sleeve 214 fixed therewith via locking mechanism 276 can begin advancing toward and into the surgical site relative to and down the inner member 218. The resistance that the surrounding tissue provides thereby prevents the tissue dilation tool 210 from quickly collapsing from the extended position to a retracted or partially retracted position after the locking mechanism 222 is depressed. Once the intermediate sleeve 216 is advanced down the inner member 218 a sufficient amount to bring the locking member 278 into alignment with recess 284 of the inner member 218, then the selective locking mechanism 276 is operable to allow limited sliding movement of the outer sleeve 214 relative to the intermediate sleeve 216.

Without the spring 20, the tissue dilation tool 210 lacks an automatic return to the fully extended position. However, when the tool 210 is held so that the tapered ends are lower than the opposite ends of the sleeves, gravity can cause the individual member or sleeve to translate to the extended position. In addition or in the alternative, motion tool 210 can cause translation to the extended position, such grasping the outer sleeve 214 and flicking the tool 210 to cause the intermediate sleeve 216 and the inner member 218 to extend therefrom. The tissue dilator 210, after use, may be removed from the system after a docking port or other tool has been inserted to retain the tissue in its dilated position. Thus, once the tool has been removed, the tool can be positioned generally vertically and the lock 222 depressed to allow the weight of the member 218 and sleeve 216 to reconfigure the tool into the fully extended position such that the member 218 and sleeve 216 slidingly move to their extended positions. It is also contemplated that a thin wire or tool may be inserted into the end of the tool opposite the tapered ends 228, 244, 258 to push the sleeves into the extended configuration. It is further contemplated that the housing 262 may be removed and the tool may be pushed manually to its extended position. In one embodiment, the inner member 218 is sized such that the back end of the inner member 218 protrudes out of the back end of the housing 262 when the instrument has been collapsed such that pushing on the back end reconfigures the instrument to the extended position. A similarly configured inner member can also be utilized in the embodiments of FIGS. 1-17 and 34-37.

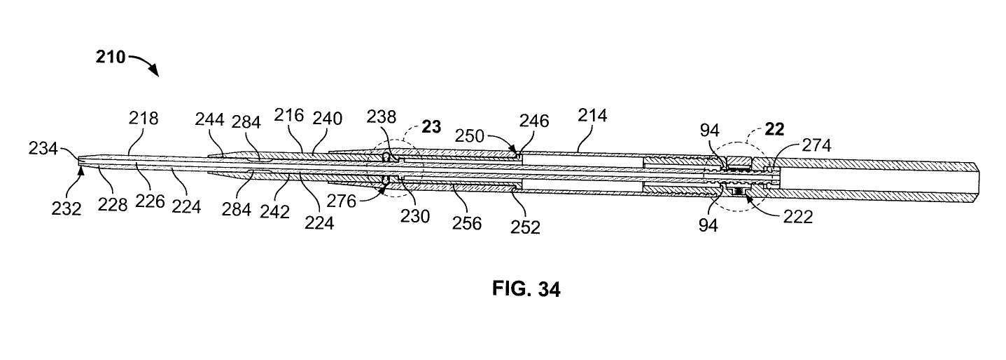

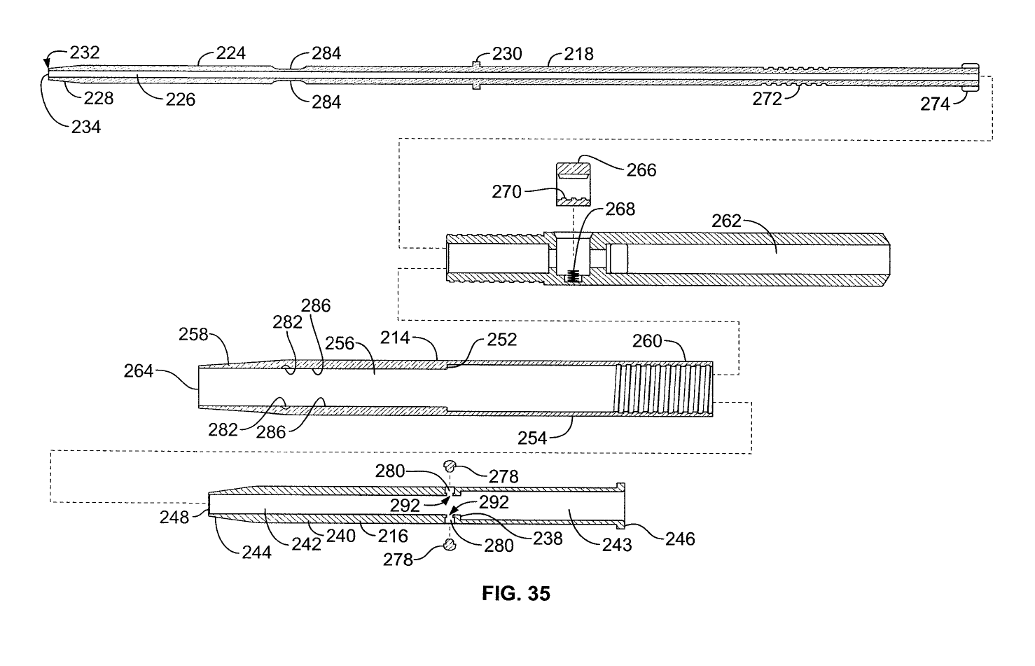

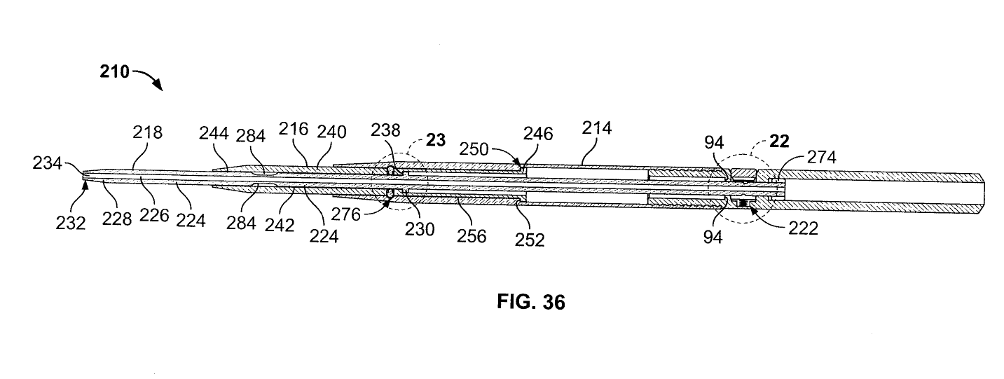

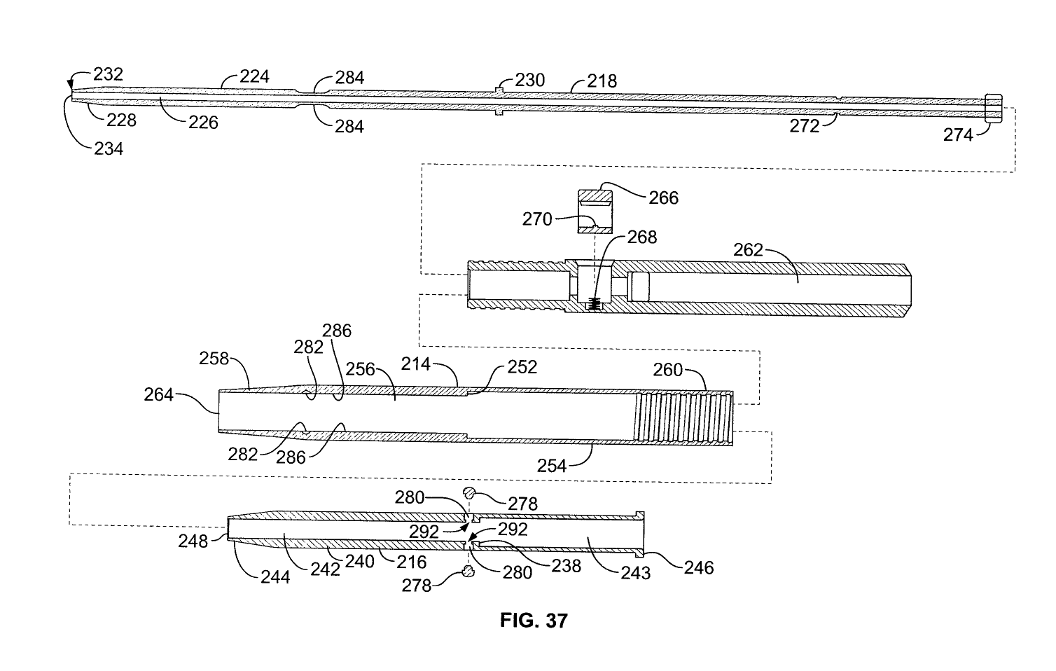

FIGS. 34-37 illustrate two additional embodiments of the tissue dilation tool 210. These two embodiments are similar to that described in FIGS. 21-33; however, the button 262 in these embodiments may be depressed momentarily and then released without stopping the members or sleeves from collapsing, whereas in the embodiment of FIGS. 21-33 the button 266 needs to be continuously depressed to permit collapse of the tool 210. The lock 222 may engage the inner sleeve 218 having a shorter threaded portion (FIGS. 34-35) or a groove or notch (FIGS. 36-37). If the inner member 218 includes a groove, as shown in FIGS. 36 and 37, the surgeon may depress the button 266 while the inner member 218 is being pushed upward into the intermediate sleeve 216 of tool 210 thereby disengaging the toothed projection 270 from the single groove 272 shown in FIG. 37. If the inner member 218 includes a short series of threads, as shown in FIGS. 34 and 35, the surgeon depresses the button 266 to begin the procedure and then continues pressing the button 266 until the inner member 218 has advanced sufficiently into the intermediate member 216 such that the threads 272 have passed the corresponding threaded projections 270 located on the button 266. Although the button 266 may be biased to a locking position with the inner member 218, the threaded projections 270 of the button 266 adjacent the inner member 218 may slide along the inner member 218 unless aligned with the groove or threads of the inner member 218.

While there have herein been illustrated and described tissue dilation tools with respect to specific examples and embodiments those skilled in the art will appreciate that there are numerous variations and permutations of the above-described apparatus and methods.

1. A tissue dilation tool comprising:
an outer sleeve;
an intermediate sleeve slidable from within the outer sleeve between an extended position and a retracted position;
an inner member slidable from within the intermediate sleeve between an extended position and a retracted position;
an inner member lock having an unlocked position where the inner member is slidable relative to the outer sleeve and a locked position where the inner member is substantially fixed against sliding relative to the outer sleeve; and
an intermediate sleeve lock having an unlocked position where the intermediate sleeve is slidable relative to the outer sleeve and a locked position where the intermediate sleeve is substantially fixed against sliding relative to the outer sleeve.

2. The tissue dilation tool of claim 1, wherein a stop is positioned between the inner member and the outer sleeve to limit the extended position of the inner member.

3. The tissue dilation tool of claim 1, wherein a stop is positioned between the intermediate sleeve and the outer sleeve to limit the extended position of the intermediate sleeve.

4. The tissue dilation tool of claim 1, wherein a stop is positioned between the inner member and the intermediate sleeve to limit the extended position of the inner member.

5. The dilation tool of claim 1, wherein the intermediate sleeve lock comprises at least one plunger positioned in an aperture of the intermediate sleeve and an inner recess of the outer sleeve to restrict sliding between the intermediate sleeve and the outer sleeve when the plunger is abutted by a larger diameter segment of the inner member, and the at least one plunger being spaced from the inner recess of the outer sleeve to permit sliding between the intermediate sleeve and the outer sleeve when the plunger is abutted by the reduced diameter segment of the inner member.

6. The dilation tool of claim 5, wherein the inner member includes a ramp between the reduced diameter and larger diameter segments of the inner member to guide the plunger into the recess of the outer sleeve as inner member slides relative to the outer member from a position where the plunger is abutted by the reduced diameter segment of the inner member to a position where the plunger is abutted by the larger diameter segment of the inner member.

7. The dilation tool of claim 1, wherein the inner member lock comprises a locking element having teeth adapted to mate with teeth formed on the exterior of the inner member, the locking element being slidable in an opening in the outer sleeve and being biased by a spring into engagement with the inner member.

8. The tissue dilation tool of claim 1, further comprising an inner spring biasing the inner member into the extended position and an intermediate spring biasing the intermediate member into the extended position, the inner spring having a reduced spring force as compared to the intermediate spring.

9. The tissue dilation tool of claim 1, further comprising an extension attached to the outer sleeve opposite a tapered end portion of the outer sleeve to block withdrawing of the inner member and intermediate sleeve from within the outer sleeve, the extension being disengageable from the outer sleeve to permit withdrawal of the inner member and intermediate sleeve from within the outer sleeve.

10. The tissue dilation tool of claim 9, wherein the extension has a bore in which a protruding end portion of the inner member is slidable when the inner member and the intermediate sleeve are in their retracted positions.

11. The tissue dilation tool of claim 1, further comprising a spring biasing the intermediate sleeve and inner member to the extended positions relative to the outer sleeve.

12. The tissue dilation tool of claim 1, wherein:
the outer sleeve includes a tapered end that the intermediate sleeve is slidable outwardly from and an opposite open end; and
the inner member includes a tapered end that is slidable outwardly from the tapered end of the outer sleeve and an opposite protruding end portion, the protruding end portion of the inner member extending outwardly through the open end of the outer sleeve when the inner member and the intermediate sleeve are in their retracted positions.

13. The tissue dilation tool of claim 1, wherein a biasing member biases the inner member lock toward the locked position and into engagement with a locking recess of the inner member when the inner member and the intermediate sleeve are in their extended positions, the inner member lock being biased against a portion of the inner member when not aligned with the locking recess to permitting sliding of the inner member against the inner member lock.

14. A method of dilating tissue surrounding an incision, the method comprising:
providing a tissue dilation tool having telescopingly arranged outer sleeve, intermediate sleeve and inner member;
locking the inner member and intermediate sleeve relative to the outer sleeve;
inserting a tapered end of the inner member into the incision to enlarge the incision to an intermediate sized opening;
unlocking the inner member relative to the outer sleeve to permit the inner member to slide within the intermediate sleeve while the intermediate sleeve is locked relative to the outer sleeve;
inserting a tapered end of the intermediate sleeve into the intermediate sized opening to enlarge the intermediate sized opening to a larger opening;
unlocking the intermediate sleeve relative to the outer sleeve to permit the intermediate member to slide within the outer sleeve; and
inserting a tapered end of the outer sleeve into the larger opening to further enlarge the opening to a final size.

15. The method of dilating tissue of claim 14, further comprising locking the inner member relative to the intermediate sleeve when the tapered end of the outer sleeve is inserted into the larger opening.

16. The method of dilating tissue of claim 14, further comprising biasing the inner member and intermediate sleeve from within the outer sleeve.

17. The method of dilating tissue of claim 14, wherein the step of inserting the tapered end of the outer sleeve further includes extending an end portion of the inner member opposite the tapered end thereof outwardly through an open end the outer sleeve opposite the tapered end thereof.

18. The method of dilating tissue of claim 17, further including the step of urging the end portion of the inner member toward the open end of the outer sleeve after the step of inserting the tapered end of the outer sleeve into the larger opening to shift the tapered end of the outer sleeve away from the tapered end of the outer sleeve.

19. The method of dilating tissue of claim 14, wherein the step of locking the inner member and intermediate sleeve relative to the outer sleeve further includes biasing a lock of the outer sleeve into engagement with a locking recess of the inner member.

20. The method of dilating tissue of claim 19, wherein the step of inserting the tapered end of the intermediate sleeve further includes permitting inner member to slide along the lock of the outer sleeve.
